(19) United States
(12) Patent Application Publication
(10) Pub. No.: US 2010/0160171 A1
(43) Pub. Date: Jun. 24, 2010

(54) Methods For Detection Of Sepsis

(75) Inventor: Robert J. FREISHTAT, Potomac, MD (US)

Correspondence Address: BLANK ROME LLP, WATERGATE, 600 NEW HAMPSHIRE AVENUE, N.W., WASHINGTON, DC, 20037, US

(73) Assignee: The Children's Research Institute, Washington, DC (US)

(21) Appl. No.: 12/644,901

(22) Filed: Dec. 22, 2009

Related U.S. Application Data
(60) Provisional application No. 61/139,936, filed on Dec. 22, 2008.

(51) Int. Cl. C40B 30/00; C12Q 1/34; G01N 33/53; C12Q 1/68

(52) U.S. Cl. 506/7; 435/18; 435/7.92; 435/6; 435/7.8

(57) ABSTRACT

The present invention relates to a method for diagnosis, detection, or prognosis of sepsis and its severity. More specifically, this invention uses the presence and amount of granzyme B in platelets as a marker for sepsis.

This application claims the priority of U.S. Provisional Patent Application Ser. No. 61/139,936, filed Dec. 22, 2008, which is incorporated herein by reference.

### FIELD OF THE INVENTION

The present invention relates to a method for diagnosis, detection, or prognosis of sepsis and its severity. More specifically, this invention uses the presence and amount of granzyme B in platelets as a marker for sepsis.

### BACKGROUND OF THE INVENTION

Despite several decades worth of advances in antimicrobials, critical care, and organ support modalities (Hotchkiss et al., N Engl J Med, 348:138-150, 2003; and Russell, N Engl J Med 355:1699-1713, 2006), mortality rates from sepsis have remained largely unchanged at about 40% (Angus et al., Crit. Care Med 29:1303-1310, 2001). In fact, sepsis is responsible for 215,000 deaths annually in the US, which is akin to mortality from acute myocardial infarction (Angus et al., 2001), making it the 10^{th} leading cause of death (Kochanek et al., Natl Vital Stat Rep 52:1-47, 2004). A recent paradigm shift indicates sepsis-related mortality results in part from immunodeficiency secondary to profound lymphoid apoptosis (Hotchkiss et al., Nat Rev Immunol 6:813-822, 2006). Indeed, this apoptosis is considered a diagnostic hallmark of progressive sepsis and multiple organ dysfunction. However, the etiology of the apoptosis is unknown.

Sepsis is characterized by a whole-body inflammatory state caused by infection. In systemic inflammations, as in the case of sepsis, the inflammation-specific reaction cascades spread in an uncontrolled manner over the whole body and become life-threatening in the context of an excessive immune response. A modern definition for sepsis given in Levy et al. (Critical Care Medicine 31(4):1250-1256, 2003).

The inflammatory processes are controlled by a large number of substances, which are predominantly of a protein or peptide nature, or are accompanied by the occurrence of certain biomolecules. The endogenous substances involved in inflammatory reactions include, particularly, cytokines, mediators, vasoactive substances, acute phase proteins and/or hormonal regulators. The inflammatory reaction is a complex physiological reaction in which both endogenous substances activating the inflammatory process (e.g. TNF-α) and deactivating substances (e.g. interleukin-10) are involved. Current knowledge about the occurrence and the possible role of individual groups of endogenous inflammation-specific substances is disclosed, for example, in Beishuizen et al. (Advances in Clinical Chemistry 33:55-131, 1999); and Gabay et al. (The New England Journal of Medicine 340(6):448-454, 1999, 448-454). Recent literature indicates sepsis-related immunodeficiency results from profound lymphoid apoptosis (Hotchkiss et al. 2003; Russell 2006; Hotchkiss et al., Scand J Infect Dis. 35(9):585-592, 2003; Groesdonk et al., J Immunol. 179(12):8083-8089, 2007; Hotchkiss et al., J. Immunol. 174(8):5110-5118, 2005; and Wesche et al., J Leukoc Biol. 78(2):325-37, 2005). Apoptosis in other end organs, such as spleen, lung, and intestine, is also common (Crouser et al., Am. J. Respir. Crit. Care Med. 161(5):1705-1712, 2000). Indeed, this apoptosis is considered a diagnostic hallmark of progressive sepsis.

For diagnostic purposes, the reliable correlation of disease with the respective biomarker is of primary importance, without there being any need to know its role in the complex cascade of the endogenous substances involved in the inflammatory process. U.S. Pat. No. 5,639,617 to Bohuon discloses the peptide procalcitonin as a marker of sepsis. U.S. Pat. No. 6,756,483 to Bergmann et al. discloses a shortened procalcitonin, containing amino acids 3-116 of the complete procalcitonin peptide, as the form that is actively involved in inflammatory processes and thus sepsis.

Other markers for sepsis include carbamoyl phosphate synthetase 1 (CPS1) or its N-terminal fragments (U.S. Pat. No. 7,413,850); CD25, CD11c, CD33, and CD66b leucocytes (U.S. Pat. No. 5,830,679); 3-chlorotyrosine or 3-bromotyrosine (U.S. Pat. No. 6,939,716); and C5aR (U.S. Pat. No. 7,455,837).

Many patients with septicemia or suspected septicemia exhibit a rapid decline over a 24-48 hour period. Thus, rapid methods of diagnosis and treatment delivery are essential for effective patient care. Clearly, there remains a need for agents capable of diagnosing and treating sepsis.

### SUMMARY OF THE INVENTION

Studies of sepsis have demonstrated accumulation of platelets in spleen and other end organs (Shibazaki et al., Infect Immun 64:5290-5294, 1996; and Drake et al., Am J Pathol 142:1458-1470, 1993). Further, activated platelet-derived microparticles have cytotoxic activity toward vascular endothelium (Azevedo et al., Endocr Metab Immune Disord Drug Targets 6:159-164, 2006; Gambim et al., Crit Care 11:R107, 2007; and Janiszewski et al., Crit Care Med 32:818-825, 2004) and smooth muscle (Janiszewski et al., 2004). However, platelets are anucleate, having only cytoplasmic components imparted by megakaryocytes residing in the bone marrow, and are incapable of de novo gene transcription. Thus, these previous studies assumed that changes in platelet function were at the post-transcriptional level. Platelets do contain reservoirs of mRNA, and a number of studies have reported the transcriptome of human platelets using mRNA profiling (Raghavachari et al., Circulation 115:1551-1562, 2007; Dittrich et al., Thromb Haemost 95:643-651, 2006; Hillmann et al., J Thromb Haemost 4:349-356, 2006; and Ouwehand et al., J Thromb Haemost 5 Suppl 1:188-195, 2007). It has also been established that platelets regulate translation of their transcriptome in response to external stimuli (Weyrich et al., Blood 109:1975-1983, 2007; Weyrich et al., Proceedings of the National Academy of Sciences 95:5556-5561, 1998; and Zimmerman et al., Arterioscler Thromb Vasc Biol 28:s17-24, 2008). However, no studies have shown acute changes in platelet mRNA pools as a function of a systemic stimulus, such as experimental or clinical sepsis.

Through genome-wide mRNA analysis, the present inventor has discovered that granzyme B is upregulated in platelets of subjects with sepsis and that the amount of granzyme B in the platelets directly corresponds to the severity of sepsis. Accordingly, this application relates to methods for the diagnosis, detection, or prognosis of sepsis, which are more sensitive and reliable than the tests of the prior art.

The present invention provides methods for detecting or diagnosing or monitoring the progression of sepsis. The methods comprise determining the presence or amount of granzyme B in platelets of an individual having or suspected of having sepsis. The presence of granzyme B (above a background level) indicates the presence of sepsis; and the amount of granzyme B directly correlates with the severity of the disease (the higher the concentration the more severe the disease).

The present invention further provides methods for monitoring the treatment of an individual with sepsis. The methods comprise administering a pharmaceutical composition to an individual suffering from sepsis, and determining the presence or amount of granzyme B in platelets of the individual. The treatment is considered successful if the amount of granzyme B decreases over the course of treatment. Treatment, however, should continue until the granzyme B amount decreases to background level or is non-detectable.

The present invention further provides methods for screening for an agent capable of modulating the onset or progression of sepsis. The methods comprise exposing an individual suffering from sepsis to the agent, and determining the presence or amount of granzyme B in platelets of the individual. The agent is considered capable of modulating the onset or progression of sepsis if, upon the administration of the agent, the amount of granzyme B decreases over the course of treatment or reduces to a background level.

In embodiments of the present invention, the amount of granzyme B is determined by detecting granzyme B gene product in platelets using immunoassays, nucleic acid analysis, preferably mRNA, or substrate degradation. Gene products as recited herein can be nucleic acid (DNA or RNA) and/or proteins. In the case of DNA and RNA, detection can occur, for example, through hybridization with oligonucleotide probes. In the case of proteins, detection can occur, for example, through various protein interaction, such as specific binding reaction (e.g. immunoassay) and substrate degradation.

A sample for granzyme B determination can be obtained by withdrawing blood from the individual. In an embodiment, the platelets in the blood sample can be lysed and the granzyme B released from the platelets can be assayed. Alternatively, the platelets can be stained using, e.g. an immunostain targeting granzyme B, and stained cells can be observed using, e.g. hemocytometry techniques known in the art. In another embodiment, the granzyme B can be detected directly from the sample.

The serum test of the present invention can be used alone or in conjunction with the other diagnostic methods known in the art, such as the markers disclosed previously in the Background of the Invention.

### BRIEF DESCRIPTION OF THE DRAWINGS

FIG. 1 shows classification of sepsis severity via unsupervised clustering of comprehensive clinical and laboratory data. Data collected over 72 hours on children and adolescents (n=17) admitted to our tertiary care pediatric ICU with a presumed diagnosis of sepsis were input into Hierarchical Clustering Explorer (HCE). Variables input included the following at 0, 24, 48, and 72 hours: Temperature; heart rate; respiratory rate; systolic, diastolic, and mean arterial blood pressure; Glasgow coma score; blood pH, pCO₂, O₂, and base excess; white blood cell count; absolute neutrophil, lymphocyte, and monocytes counts; blood hemoglobin and platelet count; prothrombin and activated partial thromboplastin times; serum sodium, potassium, chloride, glucose, creatinine; and blood urea nitrogen. Similarities between these phenotypes are reflected in the cluster shown with shorter bars representing more similarity. These results were used to classify the septic participants as severe (n=6) and moderate (n=7) as shown by the overlaid boxes.

FIG. 2 shows platelet granzyme B mRNA expression reflects megakaryocyte expression in septic mice. Platelets do not have transcriptional machinery, therefore changes in platelet granzyme B mRNA expression in septic mice (n=12) were measured simultaneously in autologous megakaryocytes. Results of this qRT-PCR analysis show good correlation between increasing megakaryocyte and platelet granzyme B mRNA expression over time.

FIG. 3 shows flow cytometric measurement of intracellular granzyme B expression in platelets from septic and healthy children. Citrated whole blood was gated on CD61⁺ platelets. Intracellular granzyme B was measured in healthy children (n=10) and septic children we classified as severe (n=1) and moderate (n=3) one and three days following admission. Shown are results from the child with severe disease showing platelet granzyme B expression at both day one (49.7%) and day three (44.3%) compared to the isotype control. Only one of the moderate septic subjects expressed any granzyme B and only at day three (24.0%). There was no measurable intracellular granzyme B in platelets from the control children.

FIG. 4 shows that platelets harvested from septic mice induce apoptosis in control CD4⁺ splenocytes except in the absence of granzyme B. Percent apoptosis was significantly higher in splenocytes co-incubated with platelets harvested from septic wild-type (i.e. C57BL6) mice than with platelets from healthy wild-type mice and splenocytes without platelets. Repeat experiments with platelets from septic granzyme B null (−/−) mice (i.e. B6.129S2-Gzmb^{tmlLey}) showed a complete lack of induced splenocyte apoptosis. Further platelet activation with recombinant TNFα under any of these conditions did not alter lymphotoxic capacity.

### DETAILED DESCRIPTION OF THE PREFERRED EMBODIMENT

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of individual genes or a group of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall (1991) Cell 64:313-326; Weirlberg (1991), Science 254:1138-1146). Thus, changes in the expression levels of particular genes or group of genes (e.g., oncogenes or tumor suppressors) serve as signposts for the presence and progression of various diseases.

Monitoring changes in gene expression may also provide certain advantages during drug screening development. Often drugs are screened and prescreened for the ability to interact with a major target without regard to other effects the drugs have on cells. Often such other effects cause toxicity in the whole animal, which prevent the development and use of the potential drug.

The present inventor has identified granzyme B in platelets as a marker associated with sepsis. Changes in granzyme B in platelets can also provide useful markers for diagnostic uses as well as markers that can be used to monitor disease states, disease progression, drug toxicity, drug efficacy and drug metabolism. Specifically, the present inventor has discovered a direct correlation between the upregulation of granzyme B in platelets and the presence of sepsis. The amount of granzyme B present directly correlates with the severity of sepsis.

### Use of Granzyme B in Platelets as Diagnostics

As described herein, the granzyme B in platelets may be used as diagnostic markers for the detection, diagnosis, or prognosis of sepsis. For instance, a sample from a patient may be assayed by any of the methods described herein or by any other method known to those skilled in the art, and the expression levels of granzyme B in platelets may be compared to the expression levels found in normal platelets (platelets in individuals without sepsis) or to the background levels of granzyme B. The expression levels of granzyme B in platelets that substantially resemble an expression level from the serum of normal or of diseased individual may be used, for instance, to aid in disease diagnosis and/or prognosis. Comparison of the granzyme B levels in platelets may be done by researcher or diagnostician or may be done with the aid of a computer and databases.

In general, the background amount of granzyme B in platelets is not detectable; thus, preferably, any detectable levels of granzyme B indicate the presence of sepsis. However, depending on the assay used, it is important to determine the background granzyme B levels to properly make a diagnosis. In general, severe sepsis is indicated if greater than about 40% of platelets express granzyme B; moderate sepsis exists if about 20-40% of platelets express granzyme B.

### Use of Granzyme B in Platelets for Drug Screening

According to the present invention, granzyme B levels in platelets may be used as markers to evaluate the effects of a candidate drug or agent on treating septic patients.

A patient suffering from sepsis is treated with a drug candidate and the progression of the disease is monitored over time. This method comprises treating the patient with an agent, periodically obtaining samples from the patient, determining the levels or amounts of granzyme B in platelets from the samples, and comparing the granzyme B levels over time to determine the effect of the agent on the progression of sepsis.

The candidate drugs or agents of the present invention can be, but are not limited to, peptides, small molecules, vitamin derivatives, as well as carbohydrates. Dominant negative proteins, DNA encoding these proteins, antibodies to these proteins, peptide fragments of these proteins or mimics of these proteins may be introduced into the patient to affect function. “Mimic” as used herein refers to the modification of a region or several regions of a peptide molecule to provide a structure chemically different from the parent peptide but topographically and functionally similar to the parent peptide (see Grant (1995), in Molecular Biology and Biotechnology, Meyers (editor) VCH Publishers). A skilled artisan can readily recognize that there is no limit as to the structural nature of the candidate drugs or agents of the present invention.

### Use of Granzyme B in Platelets for Monitoring Disease Progression

As described above, the expression of granzyme B in platelets may also be used as markers for the monitoring of disease progression, for instance, the development of sepsis. For instance, a sample from a patient may be assayed by any of the methods described herein, and the expression levels of granzyme B in platelets may be compared to the expression levels found in uninfected individuals. The levels of granzyme B in platelets can be monitored over time to track progression of the disease. The present methods are especially useful in monitoring disease progression because the granzyme B expression in platelets is proportional to the severity of the disease. Comparison of the granzyme B expression levels may be done by researcher or diagnostician or may be done with the aid of a computer and databases.

### Assay Formats

The upregulation of granzyme B in platelets is manifest at both the level of messenger ribonucleic acid (mRNA) and protein. It has been found that granzyme B in platelets, determined by either mRNA levels or biochemical measurement of protein levels, is associated with sepsis.

In an embodiment of the present invention, granzyme B levels are detected by immunoassays. Generally, immunoassays involve the binding of granzyme B and anti-granzyme B antibody. The presence and amount of binding indicate the presence and amount of granzyme B present in the sample. Examples of immunoassays include, but are not limited to, ELISAs, radioimmunoassays, immunoblots, and immunostaining, which are well known in the art. The antibody can be polyclonal or monoclonal and is preferably labeled for easy detection. The labels can be, but are not limited to biotin, fluorescent molecules, radioactive molecules, chromogenic substrates, chemiluminescence, and enzymes.

In an embodiment, ELISA, based on the capture of granzyme B by immobilized monoclonal anti-granzyme B antibody followed by detection with biotinylated polyclonal anti-granzyme B antibody, is used to detect serum granzyme B. In this system, the wells of a multi-well plate are coated with the monoclonal antibody and blocked with, e.g. milk or albumin. Samples are then added to the wells and incubated for capture of granzyme B by the monoclonal antibody. The plate may then be detected with the polyclonal antibody and strepavidine-alkaline phosphatase conjugate.

In another embodiment, granzyme B levels can be detected by measuring nucleic acid levels in the serum, preferably granzyme B mRNA. This is accomplished by hybridizing the nucleic acid, preferably at stringent conditions, in a sample with oligonucleotide probes that is specific for the granzyme B mRNA. Nucleic acid samples used in the methods and assays of the present invention may be prepared by any available method or process. Methods of isolating total RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I—Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it is desirable to inhibit or destroy RNase present in homogenates before homogenates can be used.

Nucleic acid hybridization simply involves contacting a probe and target nucleic acid under conditions where the probe and its complementary target can form stable hybrid duplexes through complementary base pairing (see U.S. Pat. No. 6,333,155 to Lockhart et al, which is incorporated herein by reference). Methods of nucleic acid hybridization are well known in the art. In a preferred embodiment, the probes are immobilized on solid supports such as beads, microarrays, or gene chips.

The hybridized nucleic acids are typically detected by detecting one or more labels attached to the sample nucleic acids and or the probes. The labels may be incorporated by any of a number of means well known to those of skill in the art (see U.S. Pat. No. 6,333,155 to Lockhart et al, which is incorporated herein by reference). Commonly employed labels include, but are not limited to, biotin, fluorescent molecules, radioactive molecules, chromogenic substrates, chemiluminescent labels, enzymes, and the like. The methods for biotinylating nucleic acids are well known in the art, as are methods for introducing fluorescent molecules and radioactive molecules into oligonucleotides and nucleotides.

Although antibodies and nucleic acid probes are specifically disclosed herein, any molecule that specifically binds granzyme B protein or mRNA can be used to detect granzyme B upregulation in manners similar to those of the antibodies or nucleic acid probes. Specific binding reactions are taught, e.g. in WO 2008/021055; and U.S. Pat. Nos. 7,321,829; 7,267,992; 7,214,346; 7,138,232; 7,153,681; 7,026,002; 6,891,057; 6,589,798; 5,939,021; 5,723,345; and 5,710,006; which are incorporated herein by reference.

Detection methods for specific binding reactions, particularly for immunoassays and the nucleic acid assays, are well known for fluorescent, radioactive, chemiluminescent, chromogenic labels, as well as other commonly used labels. Briefly, fluorescent labels can be identified and quantified most directly by their absorption and fluorescence emission wavelengths and intensity. A microscope/camera setup using a light source of the appropriate wavelength is a convenient means for detecting fluorescent label. Radioactive labels may be visualized by standard autoradiography, phosphor image analysis or CCD detector. Other detection systems are available and known in the art.

In another embodiment, because granzyme B is an enzyme, its detection can be effected through substrate degradation. In this embodiment, a sample is brought in contact with a substrate for granzyme B. The degradation of the substrate is measured which indirectly yields the levels for granzyme B. In this case, the higher the degradation rate the higher the levels of granzyme B present. Substrates for granzyme B are commercially available, e.g., through OncoImmunin, Inc., Gaithersburg, Md.; CalBiochem, San Diego, Calif.; and A.G. Scientific, Inc., San Diego, Calif. Substrates for granzyme B and their methods are disclosed, e.g., in Koeplinger, et al., Protein Exp. Purif.: 18:378, 2000; Karahashi et al., Biol. Pharm. Bull. 23:140, 2000; Harris, et al., J. Biol. Chem. 273:27364, 1998; Thornberry et al., J. Biol. Chem. 272:17907, 1997; Harris et al., J. Biol. Chem. 273:27364, 1998; and Thornberry et al., J. Biol. Chem. 272:17907, 1997; which are incorporated herein by reference. The substrates or its enzymatic products can be detected fluorometrically or colormetrically.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### Example

### Methods

Animals

Mice were purchased from Jackson Laboratories (Bar Harbor, Me., USA) and housed and bred in a conventional animal facility. All experiments were approved by our Institutional Animal Care and Use Committee. Cecal ligation and puncture was performed on male 8-12 week old mice at time=0 hours as previously described (Wichterman et al., J Surg Res 29:189-201, 1980). Briefly, under isoflurane anesthesia with spontaneous ventilation, the cecum was exposed through a 1-cm-long midline abdominal incision, ligated loosely with 4-0 silk ties (Ethicon, Cornelia, Ga., USA), and punctured twice proximally with an 18-gauge needle. Fecal material was expressed and the bowel replaced in the abdomen. The incision was closed with 4-0 nylon sutures. Mice were resuscitated with 4 ml/100 g of body weight of subcutaneous saline.

Platelet Isolation

Intra-cardiac blood was drawn directly into sodium citrate (Becton-Dickinson, Franklin Lakes, N.J., USA) and immediately centrifuged at 770 rpm for 10 minutes at 25° C. Platelets were isolated from platelet-rich plasma by a single high-speed centrifugation over Ficoll-Paque™ Plus (GE Healthcare Bio-Sciences Corporation, Piscataway, N.J., USA). Microscopy of smears of platelet isolates showed >90% platelet purity. Platelets intended for mRNA studies were immediately placed in Trizol® (Invitrogen, Carlsbad, Calif., USA). Platelets intended for functional studies were filtered through a 10 mL sepharose 2B gel column to remove extraneous proteins as described by Vollmar et al. (Microcirculation 10:143-152, 2003). Platelet concentrations were measured using a manual hemocytometer and concentrations equalized between samples by diluting with PBS.

Megakaryocyte Isolation

Murine megakaryocytes were isolated from mouse tibial and femoral bone marrow by flushing with Iscove's Modified Dulbecco's Medium (IMDM). The resulting marrow suspension was treated and passed through StemSep® magnetic gravity columns (StemCell Technologies, Vancouver, BC, Canada) according to the manufacturer's protocol using biotin-labeled anti-CD42d antibodies for positive selection. Purity was confirmed by light microscopy with Wright's stain (Sigma-Aldrich, St. Louis, Mo., USA). mRNA was isolated as described for platelets.

Splenectomy

Healthy control spleens were removed and immediately ground through a 40 μm mesh cell strainer. Splenocytes were centrifuged, washed, and layered over Ficoll-Paque™ Plus (GE Healthcare Bio-Sciences). CD4⁺ cells were isolated using StemSep® magnetic gravity columns (StemCell) according to the manufacturer's protocol.

Expression Profiling

Expression values were calculated using the dChip difference model probe set algorithm (http://biosun1.harvard.edu/complab/dchip/) and Probe Logarithmic Intensity Error Estimation (PLIER) (Affymetrix, Santa Clara, Calif.) algorithm. dChip and PLIER signals were imported into Hierarchical Clustering Explorer (HCE) (Seo et al., Bioinformatics 20:2534-2544, 2004) and the resulting unsupervised clusters were examined visually for appropriate grouping of profiles. The signals from the algorithm with the most appropriate profile grouping were used for all subsequent analyses within each species (i.e. murine=dChip, human=PLIER) and imported into GeneSpring GX (Agilent Technologies, Santa Clara, Calif., USA). The murine dataset (NCBI GEO Record #GSE10343) and human dataset (NCBI GEO Record #GSE 10361) were normalized within each chip to the 50th percentile and per gene to control chips. Using the cross-gene error model without multiple testing corrections, one-way ANOVA (p≦0.001) generated a list of differentially expressed probe sets over time.

qRT-PCR

cDNA was synthesized using the SuperScript™ III First-Strand Synthesis System (Invitrogen) per the manufacturer's protocol. DNA primers (Invitrogen) were designed according to known gene sequences as follows: granzyme A (Forward) 5′-GAA CCA CTG CTA CTC GGC ATC TGG [FAM]TC-3′; granzyme A (Reverse) 5′-CAG AAA TGT GGC TAT CCT TCA CC-3′; granzyme B (Forward) 5′-GAC GAT CCT GCT CTG ATT ACC CAT CG[FAM] C-3′; granzyme B (Reverse) 5′-TCA GAT CCT GCC ACC TGT CCT A-3′. GAPDH-containing wells served as positive controls and polymerase-free wells as negative controls. Reactions were run using an ABI PRISM® 7900HT PCR instrument (Applied Biosystems, Foster City, Calif., USA) and relative gene expression levels were calculated using Sequence Detection System 2.2 Software (Applied Biosystems). Expression values were normalized relative to sample GAPDH mRNA expression.

Detection of Apoptosis

CD4⁺ splenocytes from healthy control mice were co-incubated with platelets isolated from control or septic mice for 90 minutes at 37° C. and 5% CO₂ with or without platelet pre-treatment with 10 ng/mL of recombinant TNFα (Sigma-Aldrich) for 90 minutes. Splenocyte apoptosis was evaluated by TiterTACS™ (Trevigen, Gaithersburg, Md., USA), a quantitative colorimetric assay for in situ detection of DNA fragmentation. All samples were run in triplicate according to the manufacturer's protocol with data normalized to negative and nuclease-induced positive controls.

Statistical Analysis

Data were maintained in Microsoft Excel 2007 (Redmond, Wash., USA). Statistical significance was tested with SPSS 15 (SPSS, Chicago, Ill., USA) using paired or un-paired T-tests. Results are reported as mean±standard error of the mean (SEM) unless otherwise specified.

### Results

Sepsis Induces Platelet Cell Death Gene Expression

All mice that underwent cecal ligation and puncture (CLP) developed signs and symptoms consistent with peritoneal sepsis including decreased grooming, lethargy, and gross pathologic peritonitis at sacrifice. These mice developed significant weight loss over 48 hours (mean±SEM_{0h versus 48h}: −14.8±1.6%; p<0.0001). Fourteen out of the 96 mice studied (14.6%) expired between 6 and 48 hours status post CLP and were not included in the final analyses.

Expression profiles [Mouse 430 plus 2.0 GeneChips® (Affymetrix, Santa Clara, Calif., USA)] of platelet mRNA pooled from 5 mice at each time point (O-naïve, 24, and 48 hours status post CLP) showed 59 probe sets, representing 56 unique genes (shown in Table 1), that were differentially regulated over the time interval studied. These genes were primarily related to gene ontology biological process groups previously well-described in the response to sepsis: cell adhesion, cell growth regulation, chemotaxis, inflammatory and immune responses, proteolysis, and signal transduction. Of these, 6 probe sets belonged to the gene ontology molecular function group for cell death (GO:0008219). In particular, between 0 and 48 hours granzymes A and B, potent cytotoxic serine proteases, were >100-fold up-regulated (fold change=549.6 and 141.3 respectively).

TABLE 1
Differentially regulated probe sets (n = 59) between 0
hour controls and septic mice at 24 and 48 hours status post CLP

| | 24 Hour | 48 Hour | | | |
|---|---|---|---|---|---|
| Affymetrix | Fold | Fold | Genbank | | |
| Probe Set ID | Change | Change | ID | Gene Symbol | Gene Name |
| 1427747_a_at | 1593.0 | 540.1 | X14607 | Lcn2 | lipocalin 2 |
| 1440865_at | 276.4 | 202.3 | BB193024 | Ifitm6 | interferon induced |
| | | | | | transmembrane protein 6 |
| 1419764_at | 189.6 | 181.6 | NM_009892 | Chi3l3 | chitinase 3-like 3 |
| 1442339_at | 185.7 | 606.5 | BB667930 | MGI: 3524944 | stefin A2 like 1 |
| 1417898_{—}a_{—}at | 156.7 | 549.6 | NM_{—}010370 | Gzma | granzyme A |
| 1418809_at | 153.0 | 530.7 | NM_011087 | Pira1 | paired-Ig-like receptor A1 |
| 1449984_at | 137.5 | 206.3 | NM_009140 | Cxcl2 | chemokine (C-X-C |
| | | | | | motif) ligand 2 |
| 1451563_at | 128.4 | 1831.0 | AF396935 | Emr4 | EGF-like module |
| | | | | | containing, mucin-like, |
| | | | | | hormone receptor-like |
| | | | | | sequence 4 |
| 1456250_x_at | 126.3 | 324.6 | BB533460 | Tgfbi | transforming growth |
| | | | | | factor, beta induced |
| 1422013_at | 120.3 | 759.8 | NM_011999 | Clec4a2 | C-type lectin domain |
| | | | | | family 4, member a2 |
| 1436530_at | 109.6 | 287.0 | AA666504 | | CDNA clone |
| | | | | | MGC: 107680 |
| | | | | | IMAGE: 6766535 |
| 1450826_a_at | 104.9 | 524.0 | NM_011315 | Saa3 | serum amyloid A 3 |
| 1419394_s_at | 104.6 | 48.6 | NM_013650 | S100a8 | S100 calcium binding |
| | | | | | protein A8 (calgranulin |
| | | | | | A) |
| 1424254_at | 98.9 | 86.1 | BC027285 | Ifitm1 | interferon induced |
| | | | | | transmembrane protein 1 |
| 1442798_x_at | 93.7 | 104.1 | BB324660 | Hk3 | hexokinase 3 |
| 1456223_at | 93.2 | 246.8 | BF322016 | | Transcribed locus |
| 1416635_at | 83.0 | 683.2 | NM_020561 | Smpdl3a | sphingomyelin |
| | | | | | phosphodiesterase, |
| | | | | | acid-like 3A |
| 1437478_s_at | 81.2 | 200.3 | AA409309 | Efhd2 | EF hand domain |
| | | | | | containing 2 |
| 1422953_at | 77.8 | 62.0 | NM_008039 | Fpr-rs2 | formyl peptide receptor, |
| | | | | | related sequence 2 |
| 1436202_at | 76.1 | 160.7 | AI853644 | Malat1 | metastasis associated |
| | | | | | lung adenocarcinoma |
| | | | | | transcript 1 (non-coding RNA) |
| 1419709_at | 71.8 | 314.9 | NM_025288 | Stfa3 | stefin A3 |
| 1450808_at | 68.2 | 125.5 | NM_013521 | Fpr1 | formyl peptide receptor 1 |
| 1430700_a_at | 67.7 | 309.5 | AK005158 | Pla2g7 | phospholipase A2, |
| | | | | | group VII (platelet- |
| | | | | | activating factor |
| | | | | | acetylhydrolase, |
| | | | | | plasma) |
| 1448756_at | 67.5 | 23.5 | NM_009114 | S100a9 | S100 calcium binding |
| | | | | | protein A9 (calgranulin B) |
| 1420331_at | 65.8 | 251.8 | NM_019948 | Clec4e | C-type lectin domain |
| | | | | | family 4, member e |
| 1420330_at | 64.9 | 220.2 | NM_019948 | Clec4e | C-type lectin domain |
| | | | | | family 4, member e |
| 1423346_at | 63.2 | 272.6 | AV286991 | Degs1 | degenerative |
| | | | | | spermatocyte homolog |
| | | | | | 1 (Drosophila) |
| 1418722_at | 62.4 | 28.6 | NM_008694 | Ngp | neutrophilic granule |
| | | | | | protein |
| 1429900_at | 62.0 | 286.4 | BM241296 | 5330406M23Rik | RIKEN cDNA |
| | | | | | 5330406M23 gene |
| 1434773_a_at | 57.7 | 137.9 | BM207588 | Slc2a1 | solute carrier family 2 |
| | | | | | (facilitated glucose |
| | | | | | transporter), member 1 |
| 1420671_x_at | 57.0 | 413.4 | NM_029499 | Ms4a4c | membrane-spanning 4- |
| | | | | | domains, subfamily A, |
| | | | | | member 4C |
| 1419598_at | 55.4 | 276.6 | NM_026835 | Ms4a6d | membrane-spanning 4- |
| | | | | | domains, subfamily A, |
| | | | | | member 6D |
| 1421392_{—}a_{—}at | 53.8 | 140.5 | NM_{—}007464 | Birc3 | baculoviral IAP |
| | | | | | repeat-containing 3 |
| 1418189_s_at | 53.2 | 197.8 | AF146523 | Malat1 | metastasis associated |
| | | | | | lung adenocarcinoma |
| | | | | | transcript 1 (non-coding RNA) |
| 1435761_at | 51.2 | 322.8 | AW146083 | Stfa3 | stefin A3 |
| 1419599_s_at | 49.6 | 362.9 | NM_026835 | Ms4a11 | membrane-spanning 4- |
| | | | | | domains, subfamily A, |
| | | | | | member 11 |
| 1421408_at | 49.3 | 246.7 | NM_030691 | Igsf6 | immunoglobulin |
| | | | | | superfamily, member 6 |
| 1418204_s_at | 46.1 | 282.2 | NM_019467 | Aif1 | allograft inflammatory |
| | | | | | factor 1 |
| 1420394_s_at | 40.3 | 89.0 | U05264 | Gp49a; Lilrb4 | glycoprotein 49 A; |
| | | | | | leukocyte |
| | | | | | immunoglobulin-like |
| | | | | | receptor, subfamily B, |
| | | | | | member 4 |
| 1416530_a_at | 39.0 | 168.2 | BC003788 | Pnp | purine-nucleoside |
| | | | | | phosphorylase |
| 1437584_at | 38.8 | 158.8 | BE685667 | | Transcribed locus |
| 1419647_a_at | 38.6 | 109.6 | NM_133662 | Ier3 | immediate early |
| | | | | | response 3 |
| 1419060_{—}at | 35.2 | 141.3 | NM_{—}013542 | Gzmb | granzyme B |
| 1448123_s_at | 33.9 | 129.3 | NM_009369 | Tgfbi | transforming growth |
| | | | | | factor, beta induced |
| 1429954_at | 28.7 | 245.8 | AK014135 | Clec4a3 | C-type lectin domain |
| | | | | | family 4, member a3 |
| 1448061_at | 27.9 | 204.0 | AA183642 | Msr1 | macrophage scavenger |
| | | | | | receptor 1 |
| 1438943_x_at | 27.7 | 136.2 | AV308148 | Rpn1 | ribophorin I |
| 1439057_x_at | 23.3 | 292.2 | BB143557 | Zdhhc6 | zinc finger, DHHC |
| | | | | | domain containing 6 |
| 1448620_{—}at | 22.2 | 77.9 | NM_{—}010188 | Fcgr3 | Fc receptor, IgG, low |
| | | | | | affinity III |
| 1455899_x_at | 21.4 | 88.3 | BB241535 | Socs3 | suppressor of cytokine |
| | | | | | signaling 3 |
| 1447277_s_at | 20.9 | 630.1 | BB785407 | Pcyox1 | prenylcysteine oxidase 1 |
| 1419209_at | 20.5 | 407.7 | NM_008176 | Cxcl1 | chemokine (C-X-C |
| | | | | | motif) ligand 1 |
| 1433699_{—}at | 17.7 | 58.8 | BM241351 | Tnfaip3 | tumor necrosis factor, |
| | | | | | alpha-induced protein 3 |
| 1455908_a_at | 16.3 | 212.3 | AV102733 | Scpep1 | serine carboxypeptidase 1 |
| 1457666_s_at | 14.8 | 67.8 | AV229143 | Ifi202b | interferon activated |
| | | | | | gene 202B |
| 1427076_at | 12.9 | 91.1 | L20315 | Mpeg1 | macrophage expressed |
| | | | | | gene 1 |
| 1420249_s_at | 8.8 | 94.7 | AV084904 | Ccl6 | chemokine (C-C motif) |
| | | | | | ligand 6 |
| 1416382_at | 6.1 | 101.0 | NM_009982 | Ctsc | cathepsin C |
| 1449193_{—}at | 2.5 | 66.9 | NM_{—}009690 | Cd5l | CD5 antigen-like |

Cell Death (GO: 0008219) genes (n = 6) noted in BOLD

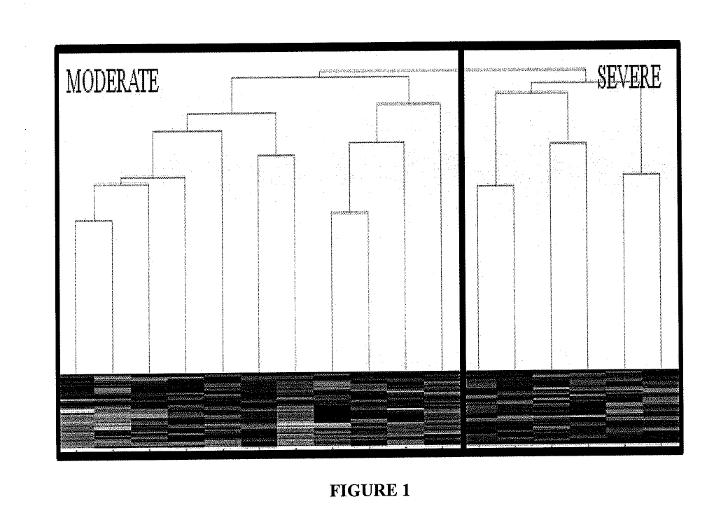

We explored expression of these cell death genes in human sepsis in an Institutional Review Board-approved study of septic children (n=17) between the ages of 1 and 18 (8.8±1.3) years. Nine participants (53%) were male. The diagnosis of sepsis was made using criteria adapted for pediatrics from the consensus definitions for sepsis (Bone et al., Chest 101:1481-1483, 1992; Proulx et al., Chest 109:1033-1037, 1996; and Proulx et al., Crit. Care Med 22:1025-1031, 1994). We collected clinical and laboratory data (i.e. the most extreme value in the prior 24 hours) over 72 hours. Relative clinical severity was determined by unsupervised clustering of all raw clinical and laboratory data in Hierarchical Clustering Explorer (HCE) (http://www.cs.umd.edu/hcil/hce/) (FIG. 1). The participants clearly clustered into two groups by clinical and laboratory variables. Group 1 (n=6) was designated “severe” because it had significantly higher severity of illness scores [i.e. mean Pediatric Risk of Mortality (PRISM) III (Pollack et al., Crit. Care Med 24:743-752, 1996) score (17.0±2.7 versus 4.5±1.1; p<0.001)] and longer hospital length of stay (45.5±10.6 versus 13.7±2.8 days; p=0.029). Group 2 (n=11) was designated “moderate” and was not significantly different from the severe group for other analyzed outcome variables including mortality and presence of shock.

As preliminary validation of the murine data, platelet mRNA from one exemplary severe and one exemplary moderate septic human subject was profiled using Human U133A GeneChips® (Affymetrix) and compared to platelet gene expression in three healthy young adult controls. There was no intent to conduct a statistically robust genome-wide assessment on this small group of samples but rather we focused on a cross-species screening for the six cell death genes identified in the murine study. Of those, only granzyme B was differentially-regulated over 72 hours (fold increase=2.9) in the severe subject. None of the other cell death genes studied showed differential expression in either group.

Validation of Sepsis-Induced Changes in the Megakaryocyte Platelet Transcriptional Axis

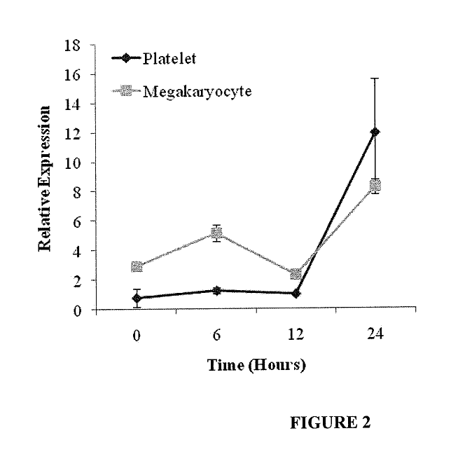

Quantitative reverse transcriptase polymerase chain reaction (qRT-PCR) was used to validate the murine platelet granzyme A and B up-regulation detected by microarray. We studied only the first 24 hours following induction of sepsis because the bulk of granzyme up-regulation seen by microarray occurred during this time period. In an independent cohort of septic mice (n=12; 3 mice per time point, non-pooled), granzyme B mRNA expression significantly increased from 0 to 24 hours (mean±SE_{0h versus 24h}: 0.77±61 versus 11.94±3.65; p=0.04) (FIG. 2). The expression of granzyme A mRNA was not significantly increased over that same time (mean±SE_{0h versus 24h} 1.57±2.73 versus 2.61±4.53; p=0.11).

As platelets are anucleate and lack transcriptional machinery, we hypothesized that increased platelet granzyme B mRNA expression in sepsis could be further validated by simultaneous measurement in autologous megakaryocytes. Using qRT-PCR we measured platelet granzyme B mRNA expression in bone marrow megakaryocytes simultaneously acquired from the same mice used in the platelet qRT-PCR validation step. Megakaryocyte granzyme B mRNA relative expression increased significantly by 24 hours (mean±SE_{0h versus 24h}: 2.88±0.27 versus 8.25±0.52; p=0.05). Platelet granzyme B mRNA expression over time closely followed that of megakaryocytes. (FIG. 2) Megakaryocyte granzyme A mRNA expression did not change (mean±SEM_{0h versus 24h}: 3.18±0.54 versus 2.99±0.12; p=0.42).

Sepsis Induces Platelet Granzyme B Protein Expression

To determine if granzyme B mRNA up-regulation translates to increased granzyme B protein expression, additional citrated whole blood was collected from septic and control mice. It was fixed with 1% paraformaldehyde, permeabilized, and intracellularly stained with anti-granzyme B (clone 16G6; eBioscience, San Diego, Calif., USA) using appropriate isotype and negative (unlabeled) controls. Flow cytometry data were generated on a FACSCalibur™ System (BD Biosciences, San Jose, Calif., USA), gating on CD61⁺ (clone 2C9.G2; BD) platelets, and analyzed using FlowJo 7.2 (Tree Star, Inc., Ashland, Oreg., USA). Platelets from septic mice (n=9) showed an increase in intracellular granzyme B protein expression after 24 hours (mean±SEM_{0h versus 24h}: 4.4±1.3 versus 19.6±6.3%; p=0.039). Additional platelet activation with tumor necrosis factor (TNF) α did not alter intracellular granzyme B (data not shown).

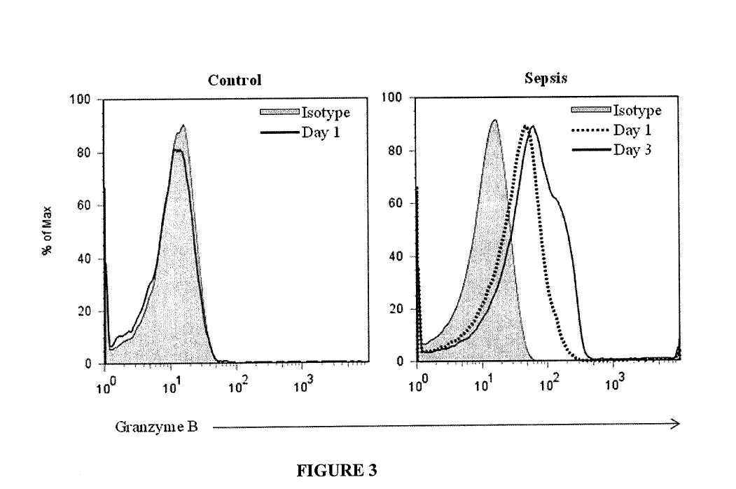

In a cross-species validation step, citrated whole blood from septic and healthy children was studied in a similar manner. In this case, flow cytometry data were generated on CD61⁺ (clone VI-PL2; BD) platelets stained for intracellular granzyme B (clone GB11; BD). Granzyme B was measured in one “severe” and three “moderate” subjects one and three days following admission for sepsis and compared to similarly-aged healthy control children (n=10) having blood drawn for routine testing. Platelets from the severe subject expressed intracellular granzyme B at both day one (49.7%) and day three (44.3%). (FIG. 3) Only one of the moderate septic subjects expressed any granzyme B and only at day three (24.0%). There was no measurable intracellular granzyme B in platelets from the control children. In addition, platelet activation state (i.e. CD62P⁺) did not affect granzyme B expression. Further, we did not detect surface expression of other apoptosis inducing proteins [i.e. Fas ligand (FasL), interleukin (IL) 1β, TNFα, and TNF-related apoptosis-inducing ligand (TRAIL)] on platelets from the septic children.

Platelets are Lymphotoxic Effectors in Sepsis Via Granzyme B

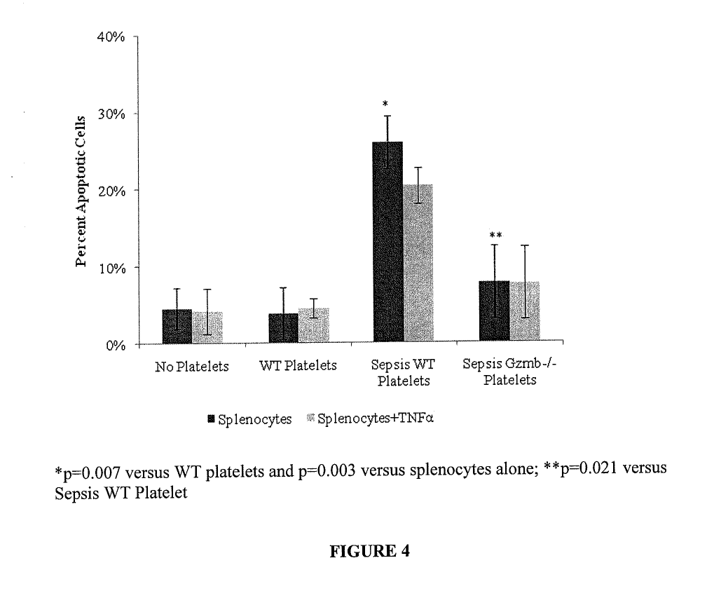

Our finding of granzyme B in platelets from septic mice and humans caused us to hypothesize that platelets could be lymphotoxic in this scenario. To study this question, platelets from mice 18 hours status post CLP were co-incubated with CD4⁺ splenocytes isolated from healthy control mice. Platelets from septic wild-type (i.e. C57BL6) mice induced marked splenocyte apoptosis compared to platelets from sham wild-type mice (rate of apoptosis=26.0±3.4 versus 3.9±3.4%; p=0.007). (FIG. 4) This co-incubation experiment was repeated with platelets from septic granzyme B null (−/−) mice (i.e. B6.129S2-Gzmb^{tmlLey}). In this case, there was a complete lack of induced splenocyte apoptosis by septic platelets. Notably, wild-type platelets further activated by TNFα had no more lymphotoxicity (4.5±1.3%; p=0.88) than non-activated control platelets. (FIG. 4)

### DISCUSSION

Sepsis-related mortality results in part from immunodeficiency secondary to profound lymphoid apoptosis (Hotchkiss et al. 2003; Russell 2006; Hotchkiss et al., Scand J Infect Dis 35:585-592, 2003; Groesdonk et al., J Immunol 179:8083-8089, 2007; Hotchkiss et al., J Immunol 174:5110-5118, 2005; and Wesche et al., J Leukoc Biol 78:325-337, 2005). The biological mechanisms responsible for this extensive lymphocyte cell death is not understood but has been attributed in part to direct pathogen signaling through toll-like receptors and MyD88 (Peck-Palmer et al. J Leukoc Biol 2008:jlb.0807528, 2008). However, in these studies we explored the possibility that platelets play a direct role in this process by conducting time series studies in a murine experimental model of sepsis. Microarrays were used as an initial screening tool to hypothesize that responses of platelets to systemic perturbations in sepsis could lead to changes in mRNA expression of cell death-associated genes. This model was then tested through a series of mouse and human studies. Our experiments led us to characterize sepsis-induced changes in the megakaryocyte-platelet transcriptional axis and present a novel finding that the resulting platelets are strongly lymphotoxic. Second, using platelets from a murine induced-sepsis model we identified the serine protease, granzyme B, as the cause of this lymphotoxicity. The granzymes are a group of cytotoxic serine proteases that are most commonly secreted within cytotoxic granules by natural killer (NK) and cytotoxic T lymphocytes (Masson et al., Cell 49:679-685, 1987). Granzyme B is the most well-characterized of these proteases (the other human granzymes include A, H, K, and M) and has multiple known caspase targets and a growing list of caspase-independent substrates, including poly(ADP-ribose) polymerase (PARP) (Froelich et al., Biochem Biophys Res Commun 227:658-665, 1996) and fibroblast growth factor receptor-1 (FGFR1) (Loeb et al., J Biol Chem 281:28326-28335, 2006). Granzyme B typically enters target cells through a channel of co-released perforin (Trapani et al., J Biol Chem 273:27934-27938, 1998) but can also enter independently (Choy et al., Arterioscler Thromb Vasc Biol 24:2245-2250, 2004; Florian et al., FEBS letters 562:87-92, 2004; and Gondek et al., J Immunol 174:1783-1786, 2005). Once in the target cell cytoplasm granzyme B cleaves several intracellular pro-apoptotic cysteine proteases, the most prominent and best-studied being caspase 3 (Trapani et al. 1998). Alternatively, granzyme B has been shown to induce apoptosis via Bid-induced mitochondrial damage (Waterhouse et al., J Biol Chem 280:4476-4482, 2005; Waterhouse et al., Cell Death Differ 13:607-618, 2006; and Waterhouse et al., Immunol Cell Biol 84:72-78, 2006). It is important to note that granzyme B has been shown to induce cell death by caspase- and non-caspase-mediated mechanisms simultaneously (Loeb et al. 2006; and Bredemeyer et al., J Biol Chem 281:37130-37141, 2006). In addition, Wong et al. showed that granzyme B is among the transcripts up-regulated in whole blood from pediatric septic shock nonsurvivors compared to survivors (Wong et al., Physiol Genomics 30:146-155, 2007).

Our experiments showed that platelets are in fact strongly lymphotoxic due to granzyme B in sepsis. Our results build upon previous research demonstrating significant inter-regulatory interactions between platelets and lymphocytes in a variety of inflammatory disease states, particularly with respect to adaptive immunity. For instance, platelet CD40 has been shown to bind to T lymphocyte CD40 ligand inducing platelet release of CCL5 which further activates T lymphocytes and thus, amplifies the immune response (Danese et al., J Immunol 172:2011-2015, 2004). In particular in sepsis, platelet-derived microparticles have been shown to be cytotoxic against vascular endothelium (Azevedo et al. 2006; Gambim et al. 2007; and Janiszewski et al. 2004) and smooth muscle (Janiszewski et al. 2004). However, to our knowledge, ours is the first study to examine acute changes in the platelet transcriptome in response to a disease insult. We found that megakaryocytes in the bone marrow respond to systemic sepsis and alter the transcriptome of platelets to include granzyme B.

The presence of granzyme B in platelets in sepsis raises intriguing questions, especially in light of the fact that platelet activation does not appear to impact its expression, implying there is no post-transcriptional regulation. First, it is possible that granzyme B serves a role in megakaryocyte caspase activation, which is critical for normal platelet formation (Clarke et al., J Cell Biol 160:577-587, 2003). If so, it is possible that in the hyper-thrombopoiesis of sepsis that megakaryocyte up-regulation of granzyme B mRNA results in inclusion of this transcript in platelets. An alternative is that platelet granzyme B represented an evolutionary advantage at some point. Granzyme B's ability to induce apoptosis through a wide variety of mechanisms makes it a likely mechanism to circumvent the immune evasion strategies of intracellular pathogens. In fact, there is evidence that granzyme B from cytotoxic T cells may play a role in defense against Toxoplasma gondii and Plasmodium species (Hurd et al., Int J Parasitol 2004; 34:1459-1472, 2004; and Gavrilescu et al., Infect Immun 71:6109-6115, 2003).

In summary, we conclude that platelets up-regulate granzyme B in murine and human sepsis. We further showed that platelets from septic mice induced marked apoptosis of healthy splenocytes ex vivo via granzyme B action. Our findings establish a conceptual advance in sepsis: Septic megakaryocytes produce platelets with acutely altered mRNA profiles and these platelets mediate lymphotoxicity via granzyme B. Given the contribution of lymphoid apoptosis to sepsis-related mortality, modulation of platelet granzyme B becomes an important new target for investigation and therapy.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A method for detecting or diagnosing or monitoring the progression of sepsis in an individual comprising the step of determining presence of granzyme B in platelets of the individual.

2. The method of claim 1, wherein the determining step is accomplished by immunoassay.

3. The method of claim 2, wherein the immunoassay is ELISA.

4. The method of claim 2, wherein in the immunoassay is an immunoblot.

5. The method of claim 1, wherein the determining step is accomplished by measuring nucleic acid levels.

6. The method of claim 4, wherein the nucleic acid is mRNA.

7. The method of claim 6, wherein the mRNA codes for granzyme B.

8. The method of claim 6, wherein the nucleic acid levels are measured by Northern blot.

9. The method of claim 6, wherein the nucleic acid levels are measured by microarray analysis.

10. The method of claim 1, wherein the determining step comprises the steps of
contacting a sample from the individual with a molecule that specifically binds the granzyme B; and
detecting the presence of binding between the granzyme B and the molecule.

11. The method of claim 10, wherein the molecule is an antibody.

12. The method of claim 11, wherein the antibody is selected from the group consisting of monoclonal antibodies and polyclonal antibodies.

13. The method of claim 10, wherein the molecule is labeled.

14. The method of claim 13, wherein the label is selected from the group consisting of biotin, fluorescent molecules, radioactive molecules, chromogenic substrates, chemiluminescence, and enzymes.

15. The method of claim 1, wherein the determining step comprises the steps of
isolating mRNA from the platelets of the individual;
contacting the isolated mRNA with a probe that specifically hybridizes with the mRNA of the granzyme B; and
detecting the presence of binding between the probe and the mRNA.

16. The method of claim 15, wherein the probe is a nucleic acid probe.

17. The method of claim 16, wherein the probe is an oligonucleotide.

18. The method of claim 16, wherein the probe is labeled.

19. The method of claim 18, wherein the label is selected from the group consisting of biotin, fluorescent molecules, radioactive molecules, chromogenic substrates, chemiluminescence, and enzymes.

20. The method of claim 15, wherein the probe is attached to a solid substrate.

21. The method of claim 15, wherein the probe is on a microarray.

22. A method for monitoring the treatment of an individual with sepsis comprising the steps of
administering a composition for treating sepsis to the individual; and
determining presence of granzyme B in platelets of the individual.

23. The method of claim 22, wherein the determining step is accomplished by immunoassay.

24. The method of claim 23, wherein the immunoassay is ELISA.

25. The method of claim 24, wherein in the immunoassay is an immunoblot.

26. The method of claim 22, wherein the determining step is accomplished by measuring nucleic acid levels.

27. The method of claim 26, wherein the nucleic acid is mRNA.

28. The method of claim 27, wherein the mRNA codes for granzyme B.

29. The method of claim 25, wherein the nucleic acid levels are measured by Northern blot.

30. The method of claim 25, wherein the nucleic acid levels are measured by microarray analysis.

31. The method of claim 24, wherein the determining step comprises the steps of
contacting the serum of the individual with a molecule that specifically binds the granzyme B; and
detecting a presence of binding between the granzyme B and the molecule.

32. The method of claim 31, wherein the molecule is an antibody.

33. The method of claim 32, wherein the antibody is selected from the group consisting of monoclonal antibodies and polyclonal antibodies.

34. The method of claim 31, wherein the molecule is labeled.

35. The method of claim 34, wherein the label is selected from the group consisting of biotin, fluorescent molecules, radioactive molecules, chromogenic substrates, chemiluminescence, and enzymes.

36. The method of claim 22, wherein the determining step comprises the steps of
isolating mRNA from the platelets;
contacting the isolated mRNA with a probe that specifically hybridize with the mRNA of the granzyme B; and
detecting a presence of binding between the probe and the mRNA.

37. The method of claim 36, wherein the probe is a nucleic acid probe.

38. The method of claim 37, wherein the probe is an oligonucleotide.

39. The method of claim 37, wherein the probe is labeled.

40. The method of claim 39, wherein the label is selected from the group consisting of biotin, fluorescent molecules, radioactive molecules, chromogenic substrates, chemiluminescence, and enzymes.

41. The method of claim 38, wherein the probe is attached to a solid substrate.

42. The method of claim 38, wherein the probe is on a microarray.

43. The method of claim 22, further comprising the step of comparing the presence of granzyme B of the individual over time to determine the effect to the composition on the progression of the sepsis.


SEQUENCE LISTING (4 sequences)

SEQ ID NO: 1 - DNA, 26 bp, Mus musculus
gaaccactgctactcggcatctggtc

SEQ ID NO: 2 - DNA, 23 bp, Mus musculus
cagaaatgtggctatccttcacc

SEQ ID NO: 3 - DNA, 27 bp, Mus musculus
gacgatcctgctctgattacccatcgc

SEQ ID NO: 4 - DNA, 22 bp, Mus musculus
tcagatcctgccacctgtccta